(19) United States
(12) Patent Application Publication
(10) Pub. No.: US 2004/0204630 A1
(43) Pub. Date: Oct. 14, 2004

(54) Device, system and method for in vivo motion detection

(76) Inventor: Zvika Gilad, Haifa (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/747,371

(22) Filed: Dec. 30, 2003

Related U.S. Application Data
(60) Provisional application No. 60/436,652, filed on Dec. 30, 2002.

(51) Int. Cl. A61B 1/00

(52) U.S. Cl. 600/160; 600/117

(57) ABSTRACT

An in-vivo device may be provided with a motion sensor unit to generate data indicative of motion of the in-vivo device. The motion detector unit may include a motion detection mechanism such as a wheel that may rotate according to in-vivo device movement, such that after such rotation may be detected, in-vivo device motion parameters may be calculated.

### CROSS REFERENCE TO RELATED APPLICATIONS

This application claims priority from U.S. Provisional Patent Application No. 60/436,652, filed Dec. 30, 2002, entitled “DEVICE, SYSTEM AND METHOD FOR IN VIVO MOTION DETECTION”, which is incorporated in its entirety herein by reference.

### FIELD OF THE INVENTION

The present invention relates to methods and devices useful in in-vivo imaging. Specifically, embodiments of the present invention relate to systems, methods and apparatuses that enable measuring the movement of in-vivo devices.

### BACKGROUND OF THE INVENTION

Ingestible in-vivo devices having an in vivo measurement system, in particular an in vivo camera system, are known in the art. For example, an in vivo video camera system may capture and transmit images of the GI tract while an in-vivo device, passes through the gastro-intestinal lumen. The system may include an in-vivo device that can pass through the entire digestive tract and operate as an autonomous video endoscope.

FIG. 1A shows an exemplary ingestible in-vivo device 110 within a human body 100. In-vivo device 110 may include an imaging device 115 at at least one end of device 110. Once inserted (e.g., swallowed), device 110 may transmit images of the gastro-intestinal (GI) tract to a set of antennas 120 within an antenna belt 125 surrounding a portion of body 100.

The GI tract moves food through it by peristaltic motion and it is this peristaltic motion that moves in-vivo device 110. However, the GI tract does not, necessarily, move matter at the same pace throughout. There are some sections that might contract softly while others might contract strongly. There might be a polyp or other protrusion that slows down the movement of food through the tract or lumens within the tract An energy management method for an in-vivo device such as device 110, is known, which senses the motion of in-vivo device 110 and switches off major power consumers, such as imaging device 115, when there is little or no motion.

### SUMMARY OF THE INVENTION

There is provided, according to embodiments of the invention, a device for detecting motion in vivo. According to one embodiment a device may include a movable element, for example, a rotatable element, and a sensor for detecting movement (e.g., rotation) of the element. According to one embodiment, the sensor may include an electromagnetic field sensor. According to another embodiment, the sensor may include an optical sensor. Other sensors and methods of sensing movement of a movable element are possible, according to further embodiments of the invention. According to one embodiment, typically in tube-like lumens, the movable element may be in contact with a body lumen wall and may thus moved by the body lumen wall when the device moves, for example, while brushing against the lumen wall. This movement may be detected by the sensor, which may indicate in-vivo device movement.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of an ingestible in-vivo device;

FIG. 2 is a schematic illustration of an in-vivo imaging system, according to some embodiments of the present invention;

FIG. 3A is a schematic illustration of a in-vivo device having a motion detector, in accordance with an embodiment of the present invention;

FIG. 3B is a cross-sectional illustration of the in-vivo device of FIG. 2A;

FIG. 4 is an alternative embodiment, shown in cross-sectional form, of a motion detector according to an embodiment of the present invention;

FIG. 5 is a flow chart illustrating a method of motion detection, according to an embodiment of the present invention, and

FIG. 6 is a flow chart illustrating an additional method of motion detection, according to an embodiment of the present invention.

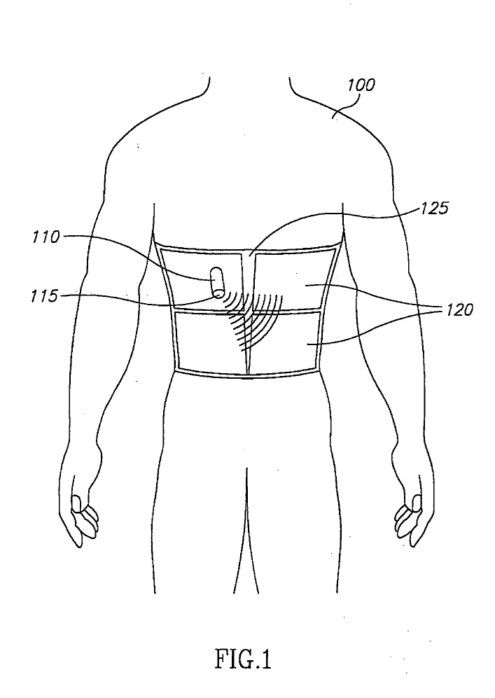

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, may refer to the action and/or processes of a processor, microprocessor, “computer on a chip”, computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the device are typically autonomous and are typically self-contained. For example, the device may be an in-vivo device or other unit where all the components are substantially contained within a container or shell, or associated with a container or shell, and where the device does not require any wires or cables to, for example, receive power or transmit information. The device may be an ingestible capsule. The device may communicate with an external receiving and display system to provide display of data, control, or other functions. For example, an internal battery or a wireless receiving system may provide power to the in-vivo device. Other embodiments may have other configurations and capabilities. For example, components may be distributed over multiple sites or units. Control information may be received from an external source.

Some embodiments of the present invention are directed to a typically swallowable in-vivo device that may be used for recording and transmitting in vivo data, for example from the entire length of the gastrointestinal (GI) tract, to a receiving and/or processing unit. Other embodiments need not be swallowable or autonomous, and may have other shapes or configurations. According to some embodiments of the present invention an in-vivo device may include at least one in-vivo sensor, for example, an image sensor, pH sensor, temperature sensor, pressure sensor, and/or other suitable sensors. Devices according to embodiments of the present invention may be similar to embodiments described in International Application WO 01/65995 and/or in U.S. Pat. No. 5,604,531, each of which are assigned to the common assignee of the present invention and each of which are hereby incorporated by reference in their entirety. Furthermore, receiving, storage, processing and/or display systems suitable for use with embodiments of the present invention may be similar to embodiments described in WO 01/65995 and/or in U.S. Pat. No. 5,604,531. Of course, devices, systems, structures, functionalities and methods as described herein may have other configurations, sets of components and processes etc.

It is noted that while a device, system and method in accordance with some embodiments of the invention may be used, for example, in a human body, the invention is not limited in this respect. For example, some embodiments of the invention may be used in conjunction or inserted into a non-human body, e.g., a dog, a cat, a rat, a cow, or other animals, pets, laboratory animals, etc.

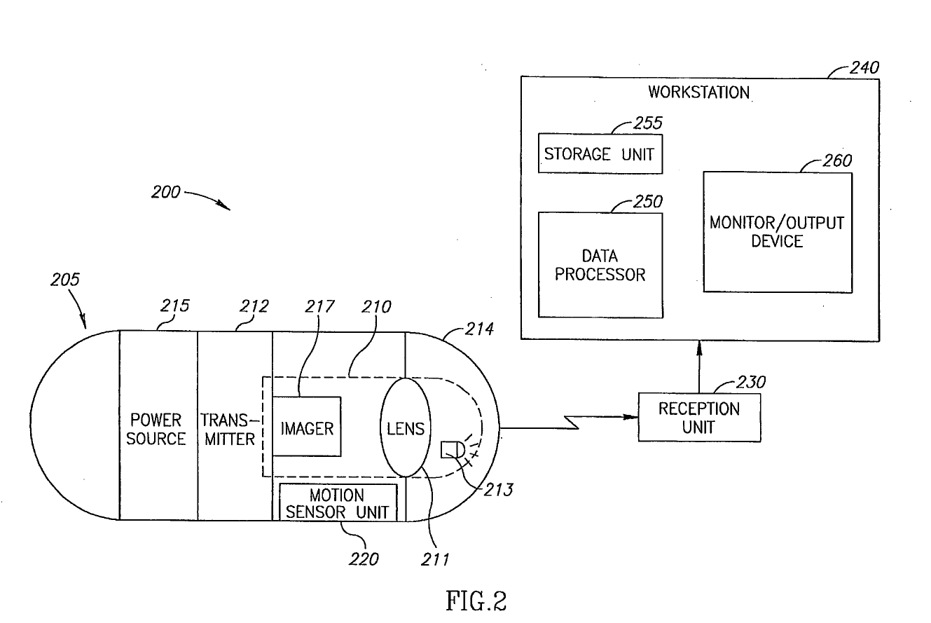

Reference is now made to FIG. 2, which is a schematic illustration of an in-vivo imaging device and system 200, according to some embodiments of the present invention. System 200 may include, for example; a swallowable in-vivo device 205, for example, an ingestible capsule, which may include an in-vivo sensing device such as an imaging module 210, which may include, for example, an optical system. Such an optical system may include, for example, a lens 211, which may focus images onto an imager 212, for example a CMOS imaging camera, a Charge Coupled Device (CCD), photodiode, or any other suitable light detector or imaging device. Illumination source 213 may illuminate the inner portions of body lumen through at least one optical window 214. In-vivo device 205 may include a transmitter 212, which may transmit, for example, radio frequency data obtained in vivo. Transmitter 212 may include, for example, a controller or processor, for example, an ASIC controller, optionally located within transmitter 212, or within any other component of device 205, to enable processing of recorded data and/or to control device 205. In-vivo device 205 may include one or more motion sensor units 220, to detect motion of in-vivo device 205 within a body. Motion sensor unit(s) 220 may include a rotatable element, for example, a wheel, a sphere, a ball, etc, wherein at least one portion of the element is distinguishable (e.g., optically distinguishable, physically distinguishable, magnetically distinguishable) from other portions of the rotatable element. In-vivo device 205 may include a power source such as, for example, a battery (e.g., a silver oxide battery, etc.) or any other suitable power source that may provide power to the electrical elements of device 205.

System 200 may include a reception unit 230, for receiving in-vivo device data (e.g., image data, movement data), and a workstation 240 to receive, process and output in-vivo device data. Workstation 240 may include a data processor 250, and displaying apparatus 260. For example, data receiver unit 230 may receive image data or other suitable data from the in-vivo imaging device 205, and may thereafter transfer the data to data processor 250, and optionally to a data storage unit 255. The data may be displayed on displaying apparatus 3200, for example, a monitor or another suitable output device. Data receiver unit 230 may be separate from the processing unit 250 or combined with it. Data processor 250 may be, for example, a personal computer or workstation. Data processor 250 may be configured for real time processing and/or for post processing to be viewed or otherwise displayed at a later date. Units 230, 250, 255 and 260 may be integrated into a single unit, or any combinations of the various units may be implemented. Of course, other suitable components may be used, as may other structures and dimensions. The in-vivo sensing device may be other than an imager, such as a pH meter, a temperature sensor, etc.

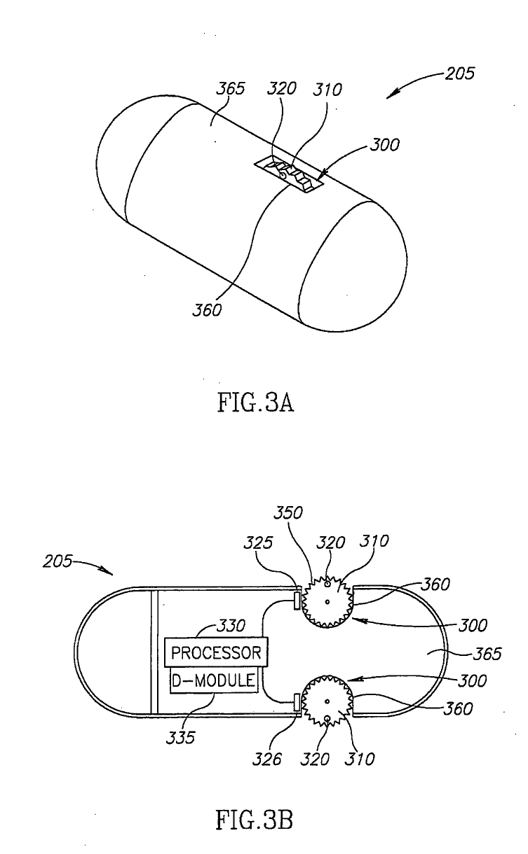

Reference is now made to FIGS. 3A and 3B which illustrate an in-vivo device 205 having one or more motion sensor unit(s) 300, in accordance with an embodiment of the present invention. Motion sensor unit(s) 300 may sense or detect the motion of in-vivo device 205 as it moves through the GI tract, for example, by having a motion detection mechanism or unit, for example, a wheel 310, which may react to movement of in-vivo device 205, for example, by being rotated as device 205 moves along an in-vivo surface. A rotatable element (e.g., wheel 310), may include at least one distinguishable portion that may be distinguished from other portions of the rotatable element. For example, the distinguishable portion may include an element that may be distinguished from other portions by appearance, texture, chemical or physical make-up, electrical or magnetic properties, etc. Motion sensor unit(s) 300 may include a sensor unit to detect movement of the rotatable element, for example, by detecting the movement or position of the distinguishable portion(s) of the rotatable element. The sensor unit may include, for example, a magnetic element, an optical sensor, mechanical sensor, electrical sensor, chemical sensor or other suitable sensor type. The movement of the rotatable element, for example, rotation, revolving or turning movements, may be translated, for example by a controller, into motion data that may include, for example, in-vivo device distance traversed, velocity, acceleration and/or other motion parameters of in-vivo device 205. Motion data may be useful, for example, in understanding and/or diagnosing in-vivo diseases and/or other conditions. Motion sensor unit 300 may also be used as the sensor for an energy management system such as that described in U.S. Pat. No. 6,428,469, which is assigned to the common assignee of the present invention and which is hereby incorporated by reference, or any other suitable energy management systems.

In the embodiment depicted in FIGS. 3A and 3B, motion sensor unit 300 of in-vivo device 205 may include at least one wheel 310 having one or more magnet(s) 320 therein, the magnet(s) being on a portion of wheel 310, such that a portion of wheel 310 with a magnet 320 may be distinguishable from portions of wheel 310 that have no magnet. In one embodiment sensor unit 300 may include, for example, one or more associated fixed magnet(s) 325, associated with magnet(s) 320, to induce a current In another embodiment, sensor unit 300 may include, for example, one or more coils 326, associated with magnet(s) 320, that may, for example, generate and send electric pulses to a counter every time that magnet 320 passes near coil 326. The current induced by magnet 320 is association with magnet 325 and/or coil 326, or another suitable detection device, may indicate the movement or positioning of magnet 320 relative to magnet 325 and/or coil 326. For example, fixed magnet 325 and/or coil 326 may have induced therein a current or a pulse of current each time that magnet 320 passes close by fixed magnet 325 and/or coil 326.

Motion sensor unit 300 may have an associated controller or processor 330. Processor 330 may be the main processor/controller of in-vivo device 205 or it may be dedicated to motion sensor 300 or other sub-systems. Another processing unit, such as the transmitter 212, may be associated with motion sensor unit 300.

In one embodiment the amount of current generated by fixed magnet 325 and/or coil 326 may be correlated, for example by performing calibration, to the amount of movement of in-vivo device 205. In another embodiment the frequency of pulses of current may be indicative of the rate of rotation of the wheel 310. Processor 330 may “count”, for example, the number of times a portion of the wheel 212, such as magnet 320, passes by a point. Knowledge of the circumference of the wheel or the physical dimensions of another sort of motion detector device may be combined with knowledge of the rate of rotation or movement to calculate distance traveled and/or velocity. This current may be transmitted, for example, via wires 322 or other suitable transmission mechanisms to processor 330. Processor 330 may receive and evaluate the current, which may help determine or indicate the movement of in-vivo device 205, and translate the current into in-vivo device motion data, for example, distance traversed or other motion parameters. Processor 330 may store the motion data, and/or may use this data to compute, for example, the velocity and/or acceleration of in-vivo device 205. Processor 330 may use the motion data to determine whether or not in-vivo device 205 has stopped moving, in which case, processor 330 may, for example, shut off or make dormant any or all of the power consuming elements of in-vivo device 205, for example, as discussed in the above mentioned U.S. Pat. No. 6,428,469. Other mode changes may be effected in response to certain movement, distance, or velocity parameters.

Processor 330 or another unit such as a transmitter 212 may include, for example, a derivative module (e.g., “D-Module”) 335, which may determine the derivative of the motion data derived from the current generated by fixed magnet 325 and/or coil 326. The derivative may be based on the distance that point (e.g., a distinguishable portion) on wheel 310 has moved, for example, the number of rounds that wheel 310 has rotated multiplied by the perimeter of wheel 310. A first derivative of the motion data may provide velocity of in-vivo device 205, for example, by calculating the distance traversed by in-vivo device 205 as a factor of a selected time interval of the measurement. A second derivative of the motion data may provide acceleration data of the in-vivo device 205, for example, by calculating the change of velocity of in-vivo device 205 as a factor of a selected time interval of the measurement. Lack of motion might be detected, for example, when there is no sign of motion (e.g. there is no electric pulse from the coil, or the velocity and/or acceleration are substantially close to zero etc.) for a pre-determined period of time. For example, the capsule may be immobile while waiting for a next peristaltic movement. For example, the ASIC in device 10 may be configured to shut off the camera after 1 minute of immobility, and turn it on after measuring movement again, to save energy. Other time intervals may be used. In alternate embodiments, processing steps such as computing derivatives, velocity, distance traveled, motion, etc., may be performed by an external processor, such as data processor 250.

Wheel(s) 310 may be made of plastic or another suitable material and may have a shape that is appropriate to enable wheel rotation when in-vivo device 205 moves along the walls of the GI tract. FIGS. 3A and 3B show wheel(s) 310 as gears with teeth 350, which may push up against the walls of lumen, channels or other in-vivo features, and may be rotated as in-vivo device 205 moves. Smooth shaped wheels may also be used, and friction-inducing mechanisms other than teeth (e.g., ridges, rough material or sticky material, etc), may be used. Other detection mechanisms may be used. Motion detection mechanisms may be moveable in other manners, other than rotating.

Wheel(s) 310 may be mounted in, for example, an indentation 360 of in-vivo device body 365 and may be exposed to the environment of the GI tract. Since wheel(s) 310 may have magnets 320 therein that correspondingly rotate with wheel(s) 310, the rotation of the wheels may induce a current in fixed magnet(s) 325. Internal components of device 205 may be isolated from the external environment, and thus a wheel well or other section containing a movement detection mechanism may be sealed from other internal portions of device 205 but open to the external environment.

The in-vivo devices exemplified in FIGS. 2 and 3 may be capsule shaped, however an in-vivo device according to embodiments of the invention may be of any shape, such as spherical, tube like and so on.

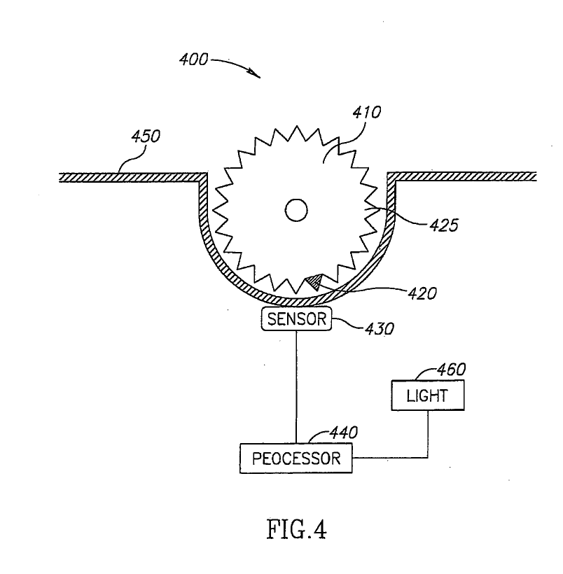

Reference is now made to FIG. 4, which illustrates a motion sensor unit 400, which may be included within or associated with an in-vivo device, in accordance with an embodiment of the present invention. Motion sensor unit 400 may include a motion detection mechanism or unit, for example, one or more rotatable elements, for example, wheel(s) 410, where the rotational element includes a portion 420 that is distinguishable from other portions 425, for example, by appearance, texture, chemical or physical make-up etc. Motion sensor unit 400 may include a sensor(s) 430, to detect movement of the rotatable element, for example, by detecting the movement or position of the distinguishable portion(s) of the rotatable element. Sensor unit(s) 430 may include, for example, a magnetic element, a coil, an optical sensor, mechanical sensor, electrical sensor, chemical sensor, or other suitable sensor type. For example, wheel 410 may have one or more different colored portions 420, for example, a differently colored tooth, ridge, section, etc. Wheel 410 may have portions distinguished other than visually, such as magnetically, by texture or raised or depressed portions, etc. As wheel(s) 410 rotates, sensor(s) 430, such as an optical sensor, may detect the wheel movement, by, for example, detecting the movement or positioning of a visibly distinguishable portion(s), and pass on data relating to such detection to processor 440. Sensor 430 may be an optical device, for example, a CMOS imaging camera, Charge Coupled Device (CCD), photodiode, imager, or any other suitable light-sensing device or imaging device.

Processor 440 may be the main processor/controller of in-vivo device 205 or it may be dedicated to motion sensor unit 400. Processor 440 may “count”, for example, the number of times portion 420 may pass by it. For example, computation by processor 440 or an alternative computational unit of the number of portion rotations in a given time period may provide velocity information of in-vivo device 10. In order for sensor(s) 430 to detect portion 420, the shell 450 of the in-vivo device may include clear plastic or another suitable transparent material. One or more portions of in-vivo device 205 may be distinguished in various ways, and may be distinguished in ways other than visually.

According to one embodiment of the present invention, portion 420 may be a substantially transparent portion. A light unit 460 may be provided that may generate a light at wheel 410. A mirror or other suitable light reflection mechanism (not seen in FIG. 4) may be placed beyond or behind transparent portion 420, such that when transparent portion 420 crosses a stream of light generated from the light unit, light may be reflected by the mirror to sensor 430. Such a reflection of light may indicate movement of wheel 410.

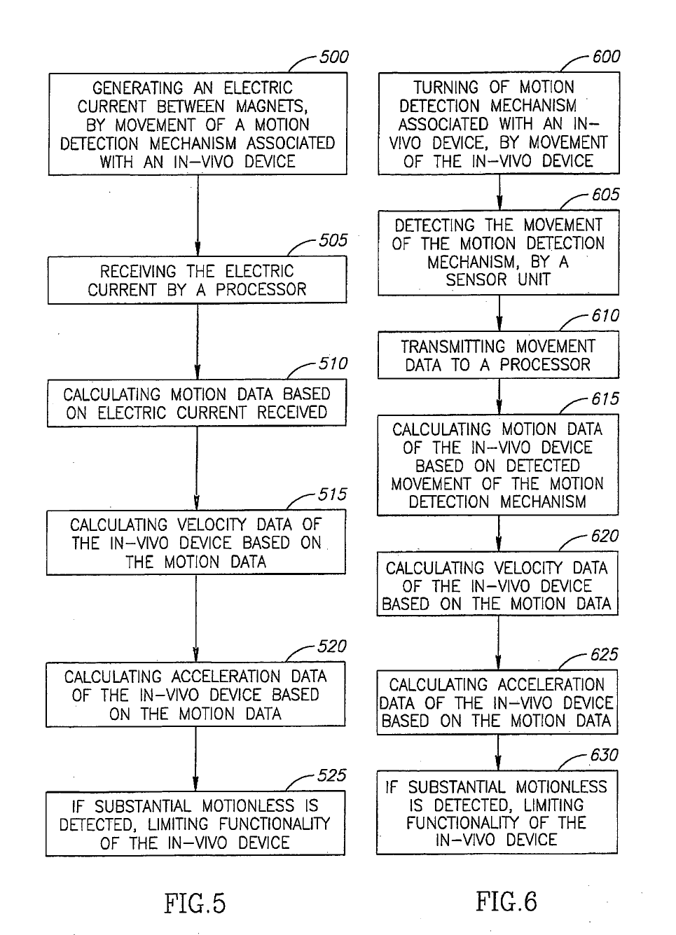

Reference is now made to FIG. 5, which illustrates a method of motion detection by in-vivo device 205, in accordance with an embodiment of the present invention. At block 500, a current between the detection unit magnets and/or the detection mechanism magnet and a coil may be generated, by movement of a motion detection mechanism associated with an in-vivo device.

At block 505, the current may be transmitted to a processor, for example, along wires or other suitable current transfer mechanisms.

At block 510, motion data may be calculated based on the electric current received, for example, according to the amount of current generated. Motion data may include, for example, in-vivo device distance traversed and/or other motion parameters.

At block 515, velocity data of the in-vivo device may be calculated, for example, based on distance traversed by the in-vivo device during a selected time period.

At block 520, acceleration data of the in-vivo device may be calculated, for example, based on the change of the in-vivo device velocity during a selected time period.

At block 525, if substantial motionless is detected, the functionality of the in-vivo device or a part of the functionality may be limited, for example, the in-vivo device imaging module may be suspended or discontinued.

Any combination of the above steps may be implemented. Further, other steps or series of steps may be used.

Reference is now made to FIG. 6, which illustrates a method of motion detection by in-vivo device 205, in accordance with an embodiment of the present invention. At block 600, a motion detection mechanism, for example a wheel, associated with an in-vivo device may be turned or rotated by movement of the in-vivo device.

At block 605, the movement of the motion detection mechanism may be detected by a sensor unit, for example, by detecting the number of times at least one distinguishable portion of the motion detection mechanism has passed the sensor.

At block 610, the detected data may be transmitted to a processor, for example, along wires or other suitable current transfer mechanisms.

At block 615, motion data of the in-vivo device may be calculated based on detected movement of the motion detection mechanism. Motion data may include, for example, in-vivo device distance traversed and/or other motion parameters.

At block 620, velocity data of the in-vivo device may be calculated based on the motion data. Motion data may include, for example, in-vivo device distance traversed and/or other motion parameters.

At block 625, acceleration data of the in-vivo device may be calculated, for example, based on the change of the in-vivo device velocity during a selected time period.

At block 630, if substantial motionless is detected, the functionality of the in-vivo device or a part of the functionality may be limited, for example, the in-vivo device imaging module may be suspended or discontinued. According to other embodiments a mode of action or operation of the in vivo device may be changed, for example, the rate of frame uptake may be increased or lowered, the illumination may be changed, or other modes may be affected.

Any combination of the above steps may be implemented. Further, other steps or series of steps may be used.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. An in-vivo device comprising:
a motion detection unit, the motion detection unit comprising a rotatable element, a portion of said element being distinguishable from other portions of said element.

2. The device of claim 1, comprising a controller to compute, from movement of said rotatable element, motion data of the in-vivo device.

3. The device of claim 2, wherein said controller is to use said motion data to calculate velocity data.

4. The device of claim 2, wherein said controller is to use said motion data to calculate acceleration data.

5. The device of claim 2, wherein said controller is to control in-vivo device functionality based on said motion data.

6. The device of claim 1, wherein said rotatable element is a wheel.

7. The device of claim 1, wherein said rotatable element comprises a magnet.

8. The device of claim 7, comprising a fixed magnet associated with said rotatable element.

9. The device of claim 7, comprising a coil associated with said rotatable element.

10. The device of claim 1, wherein said distinguishable portion is visibly distinguishable.

11. The device of claim 10, comprising a sensor to sense the movement of said visibly distinguishable portion.

12. The device of claim 1, comprising a derivative module.

13. The device of claim 1, wherein the device is an autonomous in-vivo device.

14. The device of claim 1, comprising a transmitter.

15. The device of claim 1, comprising a battery.

16. A method for in vivo motion detection, the method comprising:
generating a current between a magnet and a coil in a motion detection mechanism associated with an in-vivo device.

17. The method of claim 16, comprising calculating motion data based on said current generated.

18. The method of claim 16, comprising calculating velocity data of the in-vivo device based on distance traversed by the in-vivo device during a selected time period.

19. The method of claim 16, comprising calculating acceleration data of the in-vivo device based on change of the in-vivo device velocity during a selected time period.

20. The method of claim 16, comprising changing in-vivo device functionality if substantial motionless is detected.

21. A method comprising:
moving a motion detection mechanism associated with an in-vivo device; and
detecting movement of said motion detection mechanism.

22. The method of claim 21, comprising calculating motion data based on said movement.

23. The method of claim 21, comprising calculating velocity data of the in-vivo device based on distance traversed by the in-vivo device during a selected time period.

24. The method of claim 21, comprising calculating acceleration data of the in-vivo device based on change of the in-vivo device velocity during a selected time period.

25. The method of claim 21, comprising changing in-vivo device functionality if substantial motionless is detected.

26. The method of claim 21, comprising detecting said movement of said motion detection mechanism using a sensor to detect a distinguishable portion of said motion detection mechanism.

27. An in-vivo device comprising:
a moveable motion detector means for detecting motion of the in-vivo device; and
an in-vivo sensing means for sensing in-vivo data.

28. The device of claim 27, comprising processing means for computing motion data for the device, based on said sensed in-vivo data.
